(19) United States
(12) Patent Application Publication
(10) Pub. No.: US 2025/0140363 A1
(43) Pub. Date: May 1, 2025

(54) DATA STORAGE ON A DRUG DELIVERY DEVICE OR ON A DRUG DELIVERY ADD-ON DEVICE

(71) Applicant: Sanofi, Paris (FR)

(72) Inventor: Ronald Antony Smith, Salir (PT)

(21) Appl. No.: 18/693,870

(22) PCT Filed: Sep. 22, 2022
(86) PCT No.: PCT/EP2022/076298

§ 371 Date: Mar. 20, 2024

(30) Foreign Application Priority Data
Sep. 24, 2021 (EP) 21315178.0

(51) Int. Cl. G16H 20/17; A61M 5/31; G06F 12/14

(52) U.S. Cl. CPC G16H 20/17; G06F 12/1408; A61M 5/31; A61M 2205/52; A61M 2205/8206; G06F 2212/1052

(57) ABSTRACT

An electronic system configured for application in a drug delivery device or drug delivery add-on device to implement a data storage is disclosed. The electronic system includes at least a processor provided for processing data, a non-volatile memory provided for storing data, and an electronic component with a volatile memory, in which the electronic component is configured to be continuously supplied with electric power to maintain data stored in the volatile memory, and the processor is configured to store at least part of the processed data in the volatile memory of the electronic component.

### CROSS REFERENCE TO RELATED APPLICATIONS

The present application is the national stage entry of International Patent Application No. PCT/EP2022/076298, filed on Sep. 22, 2022, and claims priority to Application No.

EP 21315178.0, filed on Sep. 24, 2021, the disclosures of which are incorporated herein by reference.

### TECHNICAL FIELD

The present disclosure relates to data storage on a drug delivery device or on a drug delivery add-on device.

### BACKGROUND

A variety of diseases exists that require regular treatment by delivery, particularly injection of a medicament. Such injection can be performed by using injection devices, which are applied either by medical personnel or by patients themselves.

Drug delivery devices such as injection devices particularly for use by patients themselves or add-on devices for such drug delivery devices may be equipped with an electronic system for measuring and storing data related to the use. The use related data may also be transmitted via a wireless link or a wired connection to an external device such as a smartphone, a tablet or laptop computer, or to the cloud. The following documents describe drug delivery devices or add-on devices, which comprise an electronic system for measuring and storing data related to the use of the drug delivery device.

US 2019/0134305 A1 discloses a medication delivery device, for example an injection pen or a wearable pump, which can be paired with an external device for providing data captured from a flow sensor relating to medicine delivery to a patient to a paired external device. The delivery device comprises an electronic system provided integrally or via a removable attachment, for example a pen needle adapter or other pen attachment, to the delivery device. The electronic system generally comprise a processing device, a memory device, a sensor for detecting the delivery of a fluid such as the medicine or other delivery-related information, an indicator such as a light emitting diode (LED), and a communications interface,

EP 3 476 417 A1 relates to drug delivery systems for delivering, administering, injecting, infusing and/or dispensing liquids including a drug, medicament, or active ingredient. A medical monitoring system includes a disposable injection device with a container holder for holding a container or reservoir such as a cartridge or a syringe comprising a liquid drug for subcutaneous or intramuscular injection. The system also includes an electronic module or supplemental device adapted to be releasable, or reversibly, attached to the injection device. The electronic module comprises injection status sensing means for monitoring the status of an injection or for tracking progress of a medication event, as well as a tag reader different from the sensing means for reading the drug information from a machine-readable tag. The electronic module includes an evaluating unit to evaluate the drug information read from the tag, as well as a signalling unit. In this case the further evaluation information has to be available at the electronic module locally, which may imply a local clock and/or a memory unit storing a copy of the therapy plan or drug batch information in the form of a blacklist or a whitelist. The latter obviously has to be preloaded to the memory unit beforehand. The memory unit may also store information about previous medication events of the injection device.

WO2016/110592A1 relates to a wireless data communication module for a drug injection device. The wireless data communication module comprises a folded flexible carrier member comprising a plurality of stacked component support regions and a display, such as an LCD or OLED display, electrically connected to a first component support region of the folded flexible carrier member via a first set of electrical connection terminals. The display comprises an outwardly facing readable display and an opposing, downwardly facing, optical reflector. The wireless data communication module additionally comprises an NFC antenna attached to a second component support region of the folded flexible carrier member situated below the first component support region. An electronic circuit assembly of the wireless data communication module comprises at least a processor and a non-volatile memory, where the electronic circuit assembly is attached to a third component support region of the folded flexible carrier member situated below the second support region. The non-volatile memory may comprise an automatically collected data log or data record of the patient's drug administration using the drug injection device where the wireless module is integrated.

### SUMMARY

This disclosure describes solutions for data storage on a drug delivery device or on a drug delivery add-on device.

In one aspect, the present disclosure provides an electronic system configured for application in a drug delivery device or drug delivery add-on device to implement a data storage, wherein the electronic system comprises at least a processor provided for processing data, a non-volatile memory provided for storing data, and an electronic component with a volatile memory, wherein the electronic component is configured to be continuously supplied with electric power to maintain data stored in the volatile memory, and wherein the processor is configured to store at least a part of the processed data in the volatile memory of the electronic component. Instead of storing all processed data in a non-volatile memory, for example a flash memory, a part of the processed data is stored in the volatile memory of an electronic component, which is continuously supplied with electric power and, thus, behaves like non-volatile memory. Thus, erasure of that non-volatile memory can be avoided when it is full and no further processed data can be stored in it or when data must be overwritten such as changing data. Since flash memory is usually used as non-volatile memory, and flash memory requires a significant amount of electric energy for erasing, the electronic system as disclosed herein may serve to save electric energy since erasing of the non-volatile memory can be avoided, which may be an advantage for drug delivery devices and drug delivery add-on devices equipped with a primary battery or cell (a disposable and non-rechargeable battery or cell) such as the batteries or cells usually utilised in disposable injection pens.

It should be noted that the terms “memory” and “storage” are used herein as synonyms and designate the same generic technical means for storing data represented by electric charge, voltage levels, magnetic fields, electrical resistance or other electro-magnetic characteristics particularly binary digital data represented by variations in the above physical characteristics.

The term “non-volatile memory” designates technical means for storing data represented by the above mentioned electro-magnetic characteristics maintained in the absence of electric power. Examples of non-volatile memory are flash memory, read only memory (ROM), ferroelectric random access memory (RAM), and also magnetic computer storage devices such as hard disk drives. However, in the context of this application, non-volatile memory particularly means flash memory as integrated in electronic components such as microcontrollers or used in solid state drives (SSDs).

The term “volatile memory” designates technical means for storing data represented by electro-magnetic characteristics only as long as electric power is supplied. When the electric power supply of a volatile memory is turned off, the volatile memory usually loses the data stored therein.

Examples of volatile memories are static and dynamic random access memories SRAM and DRAM. However, volatile memory as used herein may also comprise internal memory of electronic components provided for storing only small amounts of data, such as registers in logic circuitry.

In an embodiment, the electronic component with the volatile memory may be a real time clock with a volatile memory being accessible by the processor, and wherein the processor is configured to access the volatile memory and to store at least the part of the processed data in the volatile memory. A real time clock usually has an internal volatile memory for storing various data, and it is also usually constantly powered to drive an oscillator and to count the oscillations generated by the driven oscillator. The requirement for electric energy to store, erase and write data and maintain stored data in the volatile memory is usually much lower than for non-volatile, but erasable and writeable memories such as flash memories. The volatile memory can be implemented as static or dynamic random access memory, and its capacity may be sufficiently large to store part of processed data.

In some embodiments, the processor may be configured to store encryption data in the volatile memory, particularly one or more encryption keys. Encryption data may change during the lifetime of a drug delivery device or drug delivery add-on device, and, thus may be suited to be stored in the volatile memory in order to avoid operations requiring more electric energy such as writing and erasing a non-volatile memory.

In further embodiments, the processor may be configured to store data related to the use of the drug delivery device, particularly data related to doses delivered with the drug delivery device, in the non-volatile memory. Use related data such as data containing information about delivered doses are normally not changed during the lifetime of the drug delivery device. Thus, this kind of data is ideally suited to be stored in the non-volatile memory as it will typically not change and thus will not need the additional electric energy required to do so.

In an embodiment, the non-volatile memory may comprise a storage size dimensioned to be sufficient for storing the entire data related to the use of the drug delivery device, particularly data related to doses delivered with the drug delivery device, generated during the lifetime of the drug delivery device. For example, depending on the drug amount stored in a cartridge of the drug delivery device and the usual dose amount per delivery, it can be determined which storage size would be sufficient to store the entire use related data over the drug delivery device's lifetime.

In yet further embodiments, the electronic system may further comprise a communication interface provided for data transmission, wherein the processor is configured to store communication related data in the volatile memory, particularly data required for establishing a data transmission to an external computing device. Such communication related data may change during the lifetime of a drug delivery device or drug delivery add-on device, and, thus may be suited to be stored in the volatile memory in order to avoid operations requiring more electric energy such as writing and erasing a non-volatile memory. The communication interface may be a wireless and/or wired communication interface, and communication related data may for example comprise data for establishing and securing a transmission with an external computing device. Particularly, the communication interface may comprise a Bluetooth® or Wi-Fi™ interface, and the communication related data may comprise Bluetooth® pairing information or a service set identifier (SSID) of a Wi-Fi™ connection.

In still further embodiments, the processor may be configured to perform at least one check and/or correction cycle with the data stored or to be stored in the volatile memory to ensure data integrity. This may ensure that the risk of data corruption when data is transferred from the processor into the volatile memory of the electronic component can be detected if not avoided. Particularly in embodiments where the connection between the processor and the volatile memory is interference-prone, for example a serial interface such as an I2C bus, data corruption could occur on such a connection during a read and/or a write event. Thus, a check cycle may be helpful to detect data corruption.

In some embodiments, the processor may be configured to perform as check cycles a checksum or hash value check, for example a Cyclic Redundancy Check (CRC), on the data before storing in the volatile memory and storing the associated checksum or hash value alongside the stored data and after reading the data and its associated checksum or hash value from the volatile memory. This is an efficient measure to quickly detect corrupted data, and may, therefore, increase confidence in the veracity of the data stored in the volatile memory.

In some embodiments, the processor may be configured to perform as check cycles multiple read operations for reading data from the volatile memory repeatedly and to compare the repeatedly read data in order to detect transient errors. Multiple read operations from the volatile memory, particularly several consecutive read operation, which require more time than a single read operation, may ensure that transient errors may be detected and corrected, which would otherwise not be possible with a single read operation.

In some embodiments, the processor may be configured to perform as check cycle a read operation of data from the volatile memory immediately after the data was stored in the volatile memory and to compare the read data with the written data in order to ensure the data was stored correctly. This is another measure to increase the integrity of data storage in the volatile memory and to ensure that no error occurred during data storage, which caused data corruption.

In some embodiments, the processor may be configured to perform as a correction cycle repeating a previous data storing operation if during a check cycle an error of the data stored during the previous data storing operation was detected. Thus, the reliability of data storing may be improved.

It should be noted that the above-described measures to increase reliability or integrity of storing data in the volatile memory and data integrity may be each implemented as single measures or they may be implemented together to improve the probability of detection of errors of the stored data.

In some embodiments, the processor may be implemented by a microcontroller and the non-volatile memory is a flash memory of the microcontroller. The microcontroller may be configured by firmware to distinguish between data to be stored in its internal flash memory, and data to be stored in external storage, particularly in the volatile memory of the electronic component, and perform storage operations according to this distinguishing of the processed data.

In further embodiments, the electronic system may comprise a primary battery or cell, particularly a button cell as a power supply, wherein the battery is mounted together with further components of the electronic system on a printed circuit board (PCB). The battery or cell is dimensioned to provide electric power over the lifetime of the drug delivery device when during the lifetime no erase operation of the non-volatile memory is carried out. The primary battery or cell may be for example soldered onto the PCB and, thus, not replaceable, as typically a disposable injection pen would not 30 have a replaceable battery or cell. The electric energy stored in the battery is particularly dimensioned to be sufficient for the expected lifetime of the drug delivery device taking into account that no erase operations are performed during that lifetime, which is possible when as disclosed herein erasable and rewriteable data are stored in the volatile memory of the electronic component.

This also allows the use of batteries or cells with a smaller capacity, i.e., batteries or cells which store less energy than would be required were erase operations required to be performed during the lifetime. Batteries or cells with smaller capacity may require less space, which may be advantageous for designing the housing of a drug delivery device or drug delivery add-on device.

In another aspect, the present disclosure provides a method for storing data on a drug delivery device or drug delivery add-on device, wherein the method comprises processing data by a processor particularly of the electronic system of any preceding claim for distinguishing between a first kind of data and a second kind of data, storing the first kind of data in a non-volatile memory and the second kind of data in a volatile memory of an electronic component being configured to be continuously supplied with electric power to maintain data stored in the volatile memory. The method may be particularly implemented as a part of firmware provided for implementing measurement of the use of a drug delivery device, particularly the delivery of doses, storing the measurement data, and providing connectivity functionality of the drug delivery device and/or drug delivery add-on device with external computing devices, for example a smartphone, a tablet computer, a laptop computer, a desktop computer, a server computer or a cloud computer.

In an embodiment of the method, the processing of data by a processor for distinguishing between a first kind of data and a second kind of data may comprise determining that a dataset among the data to be processed belongs to the first kind of data if the dataset contains data related to the use of the drug delivery device, particularly data related to doses delivered with the drug delivery device, and determining that a dataset among the data to be processed belongs to the second kind of data if the dataset contains data related communication, particularly data required for establishing a data transmission to an external computing device, and/or encryption data, particularly one or more encryption keys. This distinguishing between the first kind of data and the second kind of data may be implemented as a function of the above-mentioned firmware.

In some embodiments, the method may further comprise performing at least one check and/or correction cycle of data stored or to be stored in the volatile memory to ensure data integrity, particularly performing one or more of the following: as check cycles a checksum or hash value check on the data before storing in the volatile memory and storing the associated checksum or hash value alongside the stored data and after reading the data and its associated checksum or hash value from the volatile memory; as check cycles multiple read operations for reading data from the volatile memory repeatedly and comparing the repeatedly read data in order to detect transient errors; as a check cycle a read operation of data from the volatile memory immediately after the data was stored in the volatile memory and comparing the read data with the before data in order to ensure the data was stored correctly; as a correction cycle repeating a previous data storing operation if during a check cycle an error of the data stored during the previous data storing operation was detected. The before mentioned functions of the method may ensure data reliability and/or integrity.

### BRIEF DESCRIPTION OF THE FIGURES

FIG. 1 shows an injection device according to an embodiment;

FIG. 2 shows a schematic block diagram of an embodiment of an electronic system for application in the injection device of FIG. 1; and

FIG. 3 shows a flowchart of an embodiment of a method for storing data on the injection device of FIG. 1.

### DETAILED DESCRIPTION

In the following, embodiments of the present disclosure will be described with reference to injection devices, particularly an injection device in the form of a pen. The present disclosure is however not limited to such application and may equally well be deployed with other types of drug delivery devices, particularly with another shape than a pen. All absolute values are herein shown by way of example only and should not be construed as limiting.

An example of an injection device is an injection pen with a combined injection button and dial grip as described, e.g. in WO2014033195A1, incorporated herein by reference. Another example is an injection device with separate injection button and dial grip components as described e.g. in WO2004078239, incorporated herein by reference.

In the following discussion, the terms “distal”, “distally” and “distal end” refer to the end of an injection pen towards which a needle is provided. The terms “proximal”, “proximally” and “proximal end” refer to the opposite end of the injection device towards which an injection button or dose knob is provided.

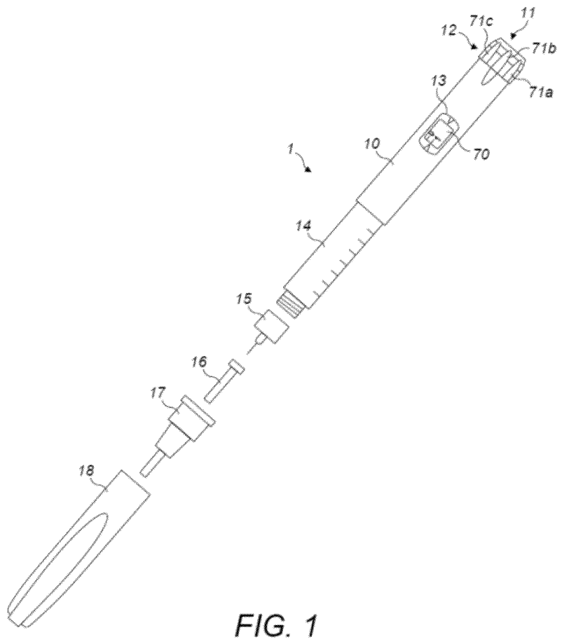

FIG. 1 is an exploded view of an injection pen 1, such as described in WO2014033195 . . . . The injection pen 1 of FIG. 1 is a pre-filled, disposable injection pen that comprises a housing 10 and contains an insulin container 14, to which a needle 15 can be affixed. The needle is protected by an inner needle cap 16 and either an outer needle cap 17 or another cap 18. An insulin dose to be ejected from injection pen 1 can be programmed, or ‘dialled in’ by turning a dose knob 12, and a currently programmed dose is then displayed via dose window 13, for instance in multiples of units. For example, where the injection pen 1 is configured to administer human insulin, the dose may be displayed in so-called International Units (IU), wherein one IU is the biological equivalent of about 45.5 micrograms of pure crystalline insulin (1/22 mg). Other units may be employed in injection devices for delivering analogue insulin or other medicaments. It should be noted that the selected dose may equally well be displayed differently than as shown in the dose window 13 in FIG. 1.

The dose window 13 may be in the form of an aperture in the housing 10, which permits a user to view a limited portion of a dial sleeve 70 that is configured to move when the dose knob 12 is turned, to provide a visual indication of a currently programmed dose. The dose knob 12 is rotated on a helical path with respect to the housing 10 when turned during programming. In this example, the dose knob 12 includes one or more formations 71a, 71b, 71c to facilitate attachment of a data collection device (drug delivery or injection add-on device).

The injection pen 1 may be configured so that turning the dose knob 12 causes a mechanical click sound to provide acoustical feedback to a user. The dial sleeve 70 mechanically inter-acts with a piston in insulin container 14. In this embodiment, the dose knob 12 also acts as an injection button. When needle 15 is stuck into a skin portion of a patient, and then dose knob 12 is pushed in an axial direction, the insulin dose displayed in display window 13 will be ejected from injection pen 1. When the needle 15 of injection pen 1 remains for a certain time in the skin portion after the dose knob 12 is pushed, a high percentage of the dose is actually injected into the patient's body. Ejection of the insulin dose may also cause a mechanical click sound, which is however different from the sounds produced when rotating the dose knob 12 during dialling of the dose.

In this embodiment, during delivery of the insulin dose, the dose knob 12 is returned to its initial position in an axial movement, without rotation, while the dial sleeve 70 is rotated to return to its initial position, e.g. to display a dose of zero units.

Injection pen 1 may be used for several injection processes until either the insulin container 14 is empty or the expiration date of the medicament in the injection pen 1 (e.g., 28 days after the first use) is reached.

Furthermore, before using injection pen 1 for the first time, it may be necessary to perform a so-called “prime shot” to remove air from insulin container 14 and needle 15, for instance by selecting two units of insulin and pressing the dose knob 12 while holding injection pen 1 with the needle 15 upwards. For simplicity of presentation, in the following, it will be assumed that the ejected amounts substantially correspond to the injected doses, so that, for instance the amount of medicament ejected from the injection pen 1 is equal to the dose received by the user. Nevertheless, differences (e.g., losses) between the ejected amounts and the injected doses may need to be taken into account.

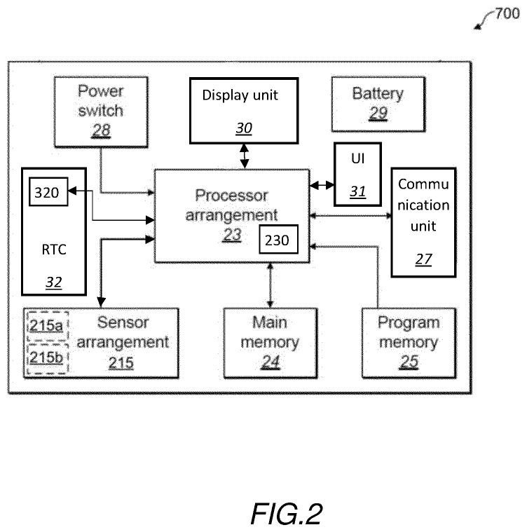

As explained above, the dose knob 12 also functions as an injection button so that the same component is used for dialling and dispensing. A sensor arrangement 215 (FIG. 2) comprising one or more optical sensors may be mounted in the injection button or dose knob 12 which is configured to sense the relative rotational position of the dial sleeve 70 relative to the injection button 12. This relative rotation can be equated to the size of the dose dispensed or delivered and used for the purpose of generating and storing or displaying dose history information. The sensor arrangement 215 may comprise a primary (optical) sensor 215a and a secondary (optical) sensor 215b. The sensor arrangement 215 may be also mounted in drug delivery or injection add-on device, which may be provided for use with different injection devices 1 and configured to collect data acquired with the sensor arrangement 215. Examples of the sensor arrangement 215 are described in detail in WO2019101962A1. The relative rotation of the dial sleeve 70 sensed by the sensor arrangement 215 with the two optical sensors 215a, 215b may be processed to determine a medicament dose administered by the injection device 1 as described below.

The sensor signal processing is performed by a sensor unit 700, as shown schematically in FIG. 2. The sensor unit 700 may be integrated in the injection device 1, particularly in the dose knob 12, or in an add-on device for attachment to the device 1. The sensor unit 700 may comprise the sensor arrangement 215 including the two sensors 215a, 215b and a device for controlling the sensor arrangement 215. The controlling device may comprise a processor arrangement 23 including one or more processors, such as a microprocessor, a Digital Signal Processor (DSP), Application Specific Integrated Circuit (ASIC), Field Programmable Gate Array (FPGA) or the like, memory units 24, 25, including program memory 24 and main memory 25, a communication unit or output 27, which may be a wireless communications interface for communicating with another device via a wireless network such as Wi-Fi™ or Bluetooth®, and/or an interface for a wired communications link, such as a Universal Series Bus (USB) socket, a display unit 30, for example a liquid crystal display (LCD), one or more LEDs, and/or an electronic paper display, a user interface (UI) 31, for example one or more buttons and/or touch input devices, a power switch 28, a primary battery or cell 29, and a real time clock (RTC) 32 for keeping track of the date and time. The RTC 32 may be implemented as a separate electronic component and comprise an internal memory 320 provided for storing time and/or date values. The primary battery or cell 29 may be a disposable, non-rechargeable, button cell, which has a capacity to store electric energy, which is dimensioned to provide electric power over the lifetime of the injection device 1 provided that no operation is carried out by the sensor unit 700 which exceeds predefined power requirements such as, for example, Flash memory erase operations.

The sensor unit 700 may comprise different types of memory: the program memory 24 may be particularly a ROM or programmable ROM (PROM) for persistently storing a firmware of the electronic system of the sensor unit 700; the main memory 25 may be particularly a DRAM or SRAM, which can store program parameters of the firmware or functions of the firmware, which may frequently change during the operation of the injection device 1 and do not need to be persistently stored and are only required during use of the injection device 1, i.e., when a drug dose is selected and delivered. The main memory 25 may particularly be powered off and lose its content once it is powered off. The sensor unit 700 may also be configured to power off the main memory 25 when the injection device 1 is not used and operated in order to save energy. The program memory 24 and/or the main memory 25 may be implemented as external components, i.e., they may be separate components and not being internal memories of devices, even though they can also be implemented as internal memories like memory 230, which is an internal memory of the processor arrangement 23. The internal memory 230 may be implemented as persistent or non-volatile, but writeable memory, particularly a flash memory, and provided to store data processed by the processor arrangement 23 and provided for storage over the lifetime of the injection device 1. Typically, administered medicament or drug doses delivered with the injection 30 device 1 are stored in the non-volatile internal memory 230, for example immediately after the processor arrangement 23 has derived the delivery drug doses from measurements received from the sensor arrangement 215. Due to the integration with other components of the processor arrangement 23 for example in a single chip, particularly a microcontroller with an integrated flash memory, the risk of data loss or corruption for example due to wiring problems or EMC (electromagnetic compatibility) issues may be reduced. The size of the non-volatile internal memory 230 may be selected such that data related to the use of the injection device 1 and produced during the lifetime of the injection device 1 can be entirely stored in the memory 230 without requiring erasing operations, which require a high electric energy to be performed. Another internal memory type may be provided in components of the sensor unit 700 such as the internal memory 320 of the RTC 32. This type of memory is usually provided to store parameters of the respective component, which are required either for the normal operation of the component or for storing parameters for as long as the respective component is supplied with electric energy. The internal memory 320 of the RTC 32 is is a volatile memory, for example an internal DRAM or SRAM or a register bank and loses stored data once the RTC 32 is powered off.

The components 23, 24, 25, 27, 28, 29, 30, 31, 32 may be soldered on a PCB containing the wiring between components. The primary battery or cell 29 may be also soldered on the PCB. The sensor arrangement 215 may be also attached to the PCB, or may be wired with the processor arrangement 23. The implementation of the sensor unit 700 depends on the drug delivery device or drug delivery add-on device, in which it should be integrated. For example, a PCB with the components 23, 24, 25, 27, 28, 29, 30, 31, 32 may be integrated in the distal end of the injection device 1, and the sensors 215a, 215b may be arranged close to the moving parts, the movement of which should be detected, and connected to the PCB via wires. At least some of the components 23, 24, 25, 27 may be also comprised by a SoC (System on Chip) or microcontroller.

The power switch 28 controls powering of the processor arrangement 23, i.e., when the power switch 28 is closed the processor arrangement 23 is activated so that it draws electric current from the battery 29. The power switch 28 may electrically separate the processor arrangement 23 from the battery or cell 29 so that the arrangement 23 is unpowered as long as the power switch 28 is not activated, or the power switch 28 may “wake-up” the processor arrangement 23 once it is activated, wherein “waking-up” means that the processor arrangement 23 is put from a kind of sleep state with a minimal power consumption into a normal operating state with a normal power consumption.

Independently from the power switch 28 state, the RTC 32 is continuously powered by the battery or cell 29, i.e. continuously supplied with electric energy from the battery or cell 29, but has a relatively low power consumption compared to the processor arrangement 23. This can be for example achieved by providing a fixed, i.e., non-interruptible power supply wiring from the battery or cell 29 to the RTC 32 on the PCB. The continuous supply with electric energy of the RTC 32 serves primarily the purpose running the real time clock to track time and maintain data stored in its internal volatile memory 320, particularly clock data required for associating dose data with a time and date, which should be set only once by a user of the injection device 1, namely upon first use of the device 1 or even earlier when it is produced in the factory. The internal memory 320 may have larger capacity than is required for storing the clock data, or the stored clock data may be reduced to have some memory space available for other purposes. The internal memory 320 is accessible from outside the RTC 32, particularly by the processor arrangement 23, which can read data from and write data into the memory 320.

Firmware stored in the program memory 25 may configure the processor arrangement 23 to control the sensor arrangement 215 such that expelling of a drug dose being delivered with the device 1 can be detected and the sensors 215a, 215b each output a sensor signal corresponding to the detected delivered drug dose. The processor arrangement 23 receives the sensor signal of each of the sensors 215a, 215b and takes readings of each sensor signal, which are processed to calculate the delivered dose. A reading may comprise for example one or more voltage samples of an analogue voltage signal of the sensor 215a, 215b. A reading may also comprise an integration of an analogue voltage signal of the sensor 215a, 215b over a certain time span. Instead of voltage signals, also electric currents, electric charges or another output signal generated by a sensor may be used for taking readings, for example frequencies of a sensor signal, frequency shifts. The readings may be taken by each sensor 215a, 215b during operation of the injection device 1 to measure the number of units dispensed by the device 1. The measuring of the number of dispensed units may comprise counting peaks of each sensor signal and deriving from the counted peaks the delivered dose.

The dose derived from measured number of dispensed units may be processed by the processor arrangement 23 by preparing datasets with the doses and particularly further data such as the time and date of delivery of the dose. The datasets may contain yet further information about delivered doses, for example the kind of drug or a drug identifier containing a unique charge number etc. These datasets should not be changed over the lifetime of the injection device 1 and, thus, safely stored. These datasets may be regarded as a first data for distinguishing it from a second data, which comprises data which may change during the lifetime of the injection device 1. The firmware configures the processor arrangement 23 to store first data in a non-volatile memory, particularly the internal flash memory 230 of the processor arrangement 23 or another non-volatile, but writeable memory comprised by the sensor unit 700 (if provided).

As mentioned above, data is distinguished between being first or second data, wherein the second data may particularly comprise changing or changeable data. The second data may particularly comprise encryption keys, which may be used for example for encrypting data transmission from the sensor unit 700 to an external computing device connected to the sensor unit 700, and/or communication related data such as data required for establishing a data transmission to an external computing device. The communication related data may for example comprise Bluetooth® pairing data and/or Wi-Fi™ connection data such as a SSID and/or a shared encryption key. The second data may also comprise credentials for accessing an external computing device such as for example a cloud service, particularly a cloud processing service.

The firmware configures the processor arrangement 23 to store the second data in the internal memory 320 of the RTC 32 and/or in internal memories of other components having volatile internal memories but being continuously supplied with electric power in order maintain data stored in their internal volatile memory. For example, when the injection device 1 is coupled by a user with his/her computing device for receiving use related data via a Bluetooth® or Wi-Fi™ direct connection, the user may set the sensor unit 700 via the UI 31 into a pairing mode, in which a first-time communication connection may be established between the sensor unit 700 and the computing device. The selected mode can be for example displayed to the user on the display unit 30. Then, the user may switch the computing device into a pairing mode, in which it can query devices in its vicinity, which are ready for pairing such as the injection device 1. In the course of pairing data related to the pairing are exchanged between both devices and stored in their internal memories to be available next time when a communication should be established without requiring a new pairing. These pairing related data may be stored by the processor arrangement 23 in the internal memory 320 of the RTC 32 in order to avoid that when these data change, for example due to a new pairing with another computing device, an erase operation of for example the internal flash memory 230 would be required, which has a higher power consumption.

Several measures may be implemented to ensure that the second data are correctly stored, and integrity is preserved, and the second data are not corruptly stored. These measures may be advantageous particularly in environments, where storing of the second data in a component external to the processor arrangement 23, particularly in the RTC 32 internal memory 320, increases the risk of data loss and corruption, for example due to wiring problems or EMC issues.

According to one measure, the processor arrangement 23 may be configured by the firmware to perform one or more check and/or correction cycles of the second data before and/or after storing in the volatile memory 320 of RTC 32. This may include a checksum or hash value check, for example a CRC on the second data, which may be performed before storing the data in the memory 320 and storing the associated checksum or hash value alongside the stored data and/or after reading the data and its associated checksum or hash value from the memory 320. A checksum check such as a CRC or a hash value check may ensure that it may be determined that an error has occurred. A check or hash value may be added to blocks of the second data before storing in the memory 320, and after reading the blocks from the memory 320, the check or hash values may be used to check whether the read data contain an error.

According to another measure, the processor arrangement 23 may be configured by the firmware to perform as check cycles multiple read operations of the second data from the volatile memory 320 of RTC 32. The read operation may be repeatedly performed in order to detect transient errors when the read data are transmitted from the memory 320 to a receiver. The repeatedly read operation may be performed subsequently, for example reading of Bluetooth® pairing data from the memory 320 may be performed 5 times, and the 5 read pairing datasets may be thereafter compared to each other in order to detect any discrepancies between the read pairing datasets. If discrepancies are detected, then a further multiple read operation sequence may be performed. This measure may be repeated until no discrepancies between the read data occur.

According to a further measure, a read operation of second data may be performed as a check cycle immediately after the second data was stored in the volatile memory 320 of the RTC 32. For example, a sequence of write and a read command may be executed by the processor arrangement 23, which writes second data in the memory 320 and directly after the termination of the writing process reads the stored data, which can then be compared with the second data still available in an internal register of the processor arrangement 23. In that way, the correctness of the stored second data can be quickly ensured.

The above described measures can be also combined in order to obtain a increase the probability to detect if an error occurred. A previous data storing operation can be repeated as a correction cycle if during a check cycle an error of the data stored during the previous data storing operation was detected. With this correction cycle, data integrity can be ensured.

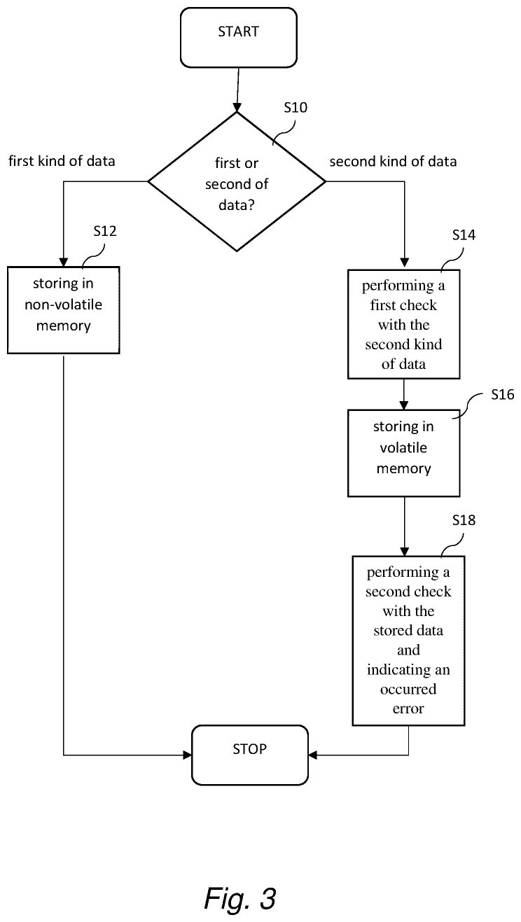

An example flowchart of a storage routine of a firmware of an injection device is shown in FIG. 3. The routine may be implemented as a procedure in the firmware, which is called when a processor requests the storage of for example processed data. The routine may then in a first step S10 distinguish the data to be stored between a first kind or a second kind of data, for example data related to delivered drug doses would be classified as first kind of data, and data related to communication aspects such as Bluetooth® pairing data would be classified as second kind of data. This distinguishing between the kind of data is used by the routine as control to decide in which memory the data are to be stored. If the data are classified as first kind of data, the routine continues with step S12 and stores the data in non-volatile memory such as the flash memory 230 of the processor arrangement 23 of the sensor unit 700 from FIG. 2. If, however, the data are classified as second kind of data, the routine continues with step S14, in which it may perform a first check with the data, for example a CRC, before it stores the checked data in step S16 in a volatile memory such as the memory 320 of the RTC 32. After having stored the data, the routine may perform in step S18 a second check with the stored data, for example read the stored data one or several times and compare the read data to detect inconsistencies or transient errors, which may have occurred during the entire storage procedure. An occurred error can then be indicated in step S18, for example by generating a corresponding signal, which may cause a user warning for example displayed on the display unit 30. The signal may also trigger a correction cycle (not shown in FIG. 3), for example by repeating the sequence of steps S14 to S18 until no error occurred in order to ensure data integrity. This correction cycle could also be manually initiated by a user, for example by making a corresponding user input via the UI 31.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or 30 mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively, or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative refers to a polypeptide which has a molecular structure which formally can be derived from the structure of a naturally occurring peptide, for example that of human insulin, in which one or more organic substituent (e.g., a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly (A21), Arg (B31), Arg (B32) human insulin (insulin glargine); Lys (B3), Glu (B29) human insulin (insulin glulisine); Lys (B28), Pro (B29) human insulin (insulin lispro); Asp (B28) human insulin (insulin aspart); human insulin, wherein proline in position B28 is replaced by Asp, Lys, Leu, Val or Ala and wherein in position B29 Lys may be replaced by Pro; Ala (B26) human insulin; Des (B28-B30) human insulin; Des (B27) human insulin and Des (B30) human insulin.

Examples of insulin derivatives are, for example, B29-N-myristoyl-des (B30) human insulin, Lys (B29) (N-tetradecanoyl)-des (B30) human insulin (insulin detemir, Levemir®); B29-N-palmitoyl-des (B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N—(N-palmitoyl-gamma-glutamyl)-des (B30) human insulin, B29-N-omega-carboxypentadecanoyl-gamma-L-glutamyl-des (B30) human insulin (insulin degludec, Tresiba®); B29-N—(N-lithocholyl-gamma-glutamyl)-des (B30) human insulin; B29-N-(ω-carboxyheptadecanoyl)-des (B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091 March-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrom.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F (ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full-length antibody polypeptide, although the term is not limited to such cleaved fragments. Antibody fragments that are useful in the present disclosure include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present disclosure, which encompass such modifications and any and all equivalents thereof.

An example drug delivery device may involve a needle-based injection system as described in Table 1 of section 5.2 of ISO 11608-1:2014(E). As described in ISO 11608-1:2014(E), needle-based injection systems may be broadly distinguished into multi-dose container systems and single-dose (with partial or full evacuation) container systems. The container may be a replaceable container or an integrated non-replaceable container.

As further described in ISO 11608-1:2014(E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1:2014(E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1:2014(E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

1-15. (canceled)

16. An electronic system configured for application in a drug delivery device or a drug delivery add-on device to implement a data storage, the electronic system comprising:
a processor configured to process data,
a non-volatile memory configured to store data, and
an electronic component comprising a volatile memory,
wherein the electronic component is configured to be continuously supplied with electric power to maintain data stored in the volatile memory, and
wherein the processor is configured to store at least a part of processed data in the volatile memory of the electronic component.

17. The electronic system of claim 16, wherein the electronic component with the volatile memory is a real time clock with the volatile memory being accessible by the processor, and wherein the processor is configured to access the volatile memory and to store at least part of the processed data in the volatile memory.

18. The electronic system of claim 16, wherein the processor is configured to store encryption data in the volatile memory.

19. The electronic system of claim 18, wherein the encryption data includes one or more encryption keys.

20. The electronic system of claim 16, wherein the processor is configured to store data related to a use of the drug delivery device in the non-volatile memory.

21. The electronic system of claim 20, wherein the data related to the use of the drug delivery device includes data related to doses delivered with the drug delivery device.

22. The electronic system of claim 20, wherein the non-volatile memory has a storage size sufficient for storing entire data related to the use of the drug delivery device, the entire data comprising data related to doses delivered with the drug delivery device and generated during a lifetime of the drug delivery device.

23. The electronic system of claim 16, further comprising a communication interface for data transmission, wherein the processor is configured to store communication related data in the volatile memory.

24. The electronic system of claim 23, wherein the communication related data comprises data required for establishing a data transmission from the electronic system to an external computing device.

25. The electronic system of claim 16, comprising a primary battery or a cell as a power supply, wherein the primary battery or the cell is mounted together with other components of the electronic system on a printed circuit board and dimensioned to provide electric power over the lifetime of the drug delivery device when, during the lifetime, no erase operation of the non-volatile memory is carried out.

26. The electronic system of claim 16, wherein the processor is configured to perform at least one check and/or correction cycle of the data stored or to be stored in the volatile memory to ensure data integrity.

27. The electronic system of claim 26, wherein the processor is configured to perform as check cycles a checksum or hash value check on the data before storing the data in the volatile memory and storing the checksum or hash value alongside the stored data and after reading the data and the checksum or hash value from the volatile memory.

28. The electronic system of claim 26, wherein the processor is configured to perform as check cycles multiple read operations for reading data from the volatile memory repeatedly and to compare the repeatedly read data in order to detect transient errors.

29. The electronic system of claim 26, wherein the processor is configured to perform as a check cycle a read operation of data from the volatile memory after the data was stored in the volatile memory and to compare the read data with the written data in order to ensure the data was stored correctly.

30. The electronic system of claim 26, wherein the processor is configured to perform as a correction cycle repeating a previous data storing operation if, during a check cycle, an error of the data stored during the previous data storing operation was detected.

31. A computer-implemented method for storing data on a drug delivery device or a drug delivery add-on device, the method comprising:
processing data by a processor of an electronic system for distinguishing between a first kind of data and a second kind of data, wherein the electronic system is configured for application in the drug delivery device or the drug delivery add-on device, wherein the electronic system comprises:
a processor configured to process data,
a non-volatile memory configured to store data, and
an electronic component comprising a volatile memory,
wherein the electronic component is configured to be continuously supplied with electric power to maintain data stored in the volatile memory, and
wherein the processor is configured to store at least a part of processed data in the volatile memory of the electronic component, and storing the first kind of data in a non-volatile memory and the second kind of data in a volatile memory of the electronic component.

32. The computer-implemented method of claim 31, wherein the processing of data by the processor for distinguishing between a first kind of data and a second kind of data comprises:
determining that a dataset among the data to be processed belongs to the first kind of data if the dataset contains data related to a use of the drug delivery device, and
determining that a dataset among the data to be processed belongs to the second kind of data if the dataset contains data related to communication and/or encryption data.

33. The computer-implemented method of claim 32, wherein the data related to the use of the drug delivery device comprises data related to doses delivered with the drug delivery device, wherein the data related to communication comprises data required for establishing a data transmission from the electronic device to an external computing device, and the encryption data comprises one or more encryption keys.

34. The computer-implemented method of claim 31, further comprising:
performing at least one check and/or correction cycle of the data stored or to be stored in the volatile memory to ensure data integrity, comprising performing one or more of the following:
as check cycles a checksum or hash value check on the data before storing the data in the volatile memory and storing the associated checksum or hash value alongside the stored data and after reading the data and its associated checksum or hash value from the volatile memory;
as check cycles multiple read operations for reading data from the volatile memory repeatedly and comparing the repeatedly read data in order to detect transient errors;
as a check cycle a read operation of data from the volatile memory after the data was stored in the volatile memory and comparing the read data with the written data in order to ensure the data was stored correctly; or
as a correction cycle repeating a previous data storing operation if, during a check cycle, an error of the data stored during the previous data storing operation was detected.

35. The computer-implemented method of claim 31, wherein the electronic component is a real time clock with the volatile memory accessible by the processor, and wherein the processor is configured to access the volatile memory and to store at least part of the processed data in the volatile memory.
